(19) United States
(12) Patent Application Publication
(10) Pub. No.: US 2008/0153858 A1
(43) Pub. Date: Jun. 26, 2008

(54) ANTITUMORIGENIC DRUG COMBINATION

(75) Inventors: David J. Frost, Deerfield, IL (US); Anne E. Hagey, Lake Forest, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 11/958,880

(22) Filed: Feb. 25, 2008

Related U.S. Application Data
(60) Provisional application No. 60/871,540, filed on Dec. 22, 2006.

(51) Int. Cl. A61K 31/519; A61K 31/44

(52) U.S. Cl. 514/265.1; 514/352

(57) ABSTRACT

This invention relates to compositions and methods for treating cancer comprising therapeutically effective amounts of ABT-751 and pemetrexed.

This application claims priority from U.S. Provisional Patent Application Ser. No. 60/871,540 filed Dec. 22, 2006, which is hereby incorporated by reference in its entirety.

### FIELD OF THE INVENTION

The invention relates to compositions comprising drugs having additive antitumorigenesis activity and methods of treatment using the combinations.

### BACKGROUND OF THE INVENTION

Neoplastic diseases are characterized by the proliferation of cells which are not subject to normal cell growth and are a major cause of death in humans and other mammals. Cancer chemotherapy has provided new and effective drugs for treating these diseases and has also demonstrated that drugs which disrupt the microtubule system of the cytoskeleton are effective in inhibiting the proliferation of neoplastic cells. Accordingly, drugs which disrupt the microtubule system are some of the most effective cancer chemotherapeutic agents in use. There exists a need for new orally-bioavailable microtubule-disrupting drug treatments with improved side-effect profiles.

### BRIEF DESCRIPTION OF THE FIGURES

FIG. 1 shows additive antitumor activity of ABT-751 and pemetrexed combination in the Calu-6 NSCLC xenograft model using a sequential schedule (pemetrexed first). Student t-test (two-sided).

FIG. 2 shows additive activity of ABT-751 and pemetrexed in a thymidine kinase minus human colon carcinoma xenograft model (GC3 TK-) using a concurrent drug schedule.

### SUMMARY OF THE INVENTION

Accordingly, one embodiment of this invention pertains to compositions for treating cancer in a mammal, said compositions comprising therapeutically effective amounts of pemetrexed and N-(2-((4-hydroxyphenyl)amino)pyridin-3-yl)-4-methoxybenzenesulfonamide.

Another embodiment of this invention pertains to compositions for treating cancer in a mammal, said compositions comprising therapeutically effective amounts of an antimetabolite and N-(2-((4-hydroxyphenyl)amino)pyridin-3-yl)-4-methoxybenzenesulfonamide.

Another embodiment pertains to separate compositions to be administered together for cancer in a mammal, one composition comprising a therapeutically effective amount of pemetrexed and the other comprising a therapeutically effective amount of N-(2-((4-hydroxyphenyl)amino)pyridin-3-yI)-4-methoxybenzenesulfonamide.

Another embodiment pertains to methods of treating cancer in a human, said methods comprising administering thereto therapeutically effective amounts of an antimetabolite and N-(2-((4-hydroxyphenyl)amino)pyridin-3-yl)-4-methoxybenzenesulfonamide.

Another embodiment pertains to methods of treating cancer in a human, said methods comprising administering thereto therapeutically effective amounts of pemetrexed and N-(2-((4-hydroxyphenyl)amino)pyridin-3-yl)-4-methoxybenzenesulfonamide.

Still another embodiment pertains to methods for treating cancer with at least additive antitumorigenesis in a mammal, said methods comprising administering thereto therapeutically effective amounts of pemetrexed and N-(2-((4-hydroxyphenyl)amino)pyridin-3-yl)-4-methoxybenzenesulfonamide.

Still another embodiment pertains to methods for treating cancer with greater than additive antitumorigenesis in a mammal, said methods comprising administering thereto therapeutically effective amounts of pemetrexed and N-(2-((4-hydroxyphenyl)amino)pyridin-3-yl)-4-methoxybenzenesulfonamide.

Still another embodiment pertains to separate compositions to be administered together for treating prostate cancer in a mammal, one composition comprising a therapeutically effective amount of pemetrexed and the other comprising a therapeutically effective amount of N-(2-((4-hydroxyphenyl)amino)pyridin-3-yl)-4-methoxybenzenesulfonamide.

Still another embodiment pertains to separate compositions to be administered together for treating lung cancer in a mammal, one composition comprising a therapeutically effective amount of pemetrexed and the other comprising a therapeutically effective amount of N-(2-((4-hydroxyphenyl)amino)pyridin-3-yl)-4-methoxybenzenesulfonamide. In another embodiment, the lung cancer is non-small cell lung cancer.

Still another embodiment pertains to separate compositions to be administered together for colon cancer in a mammal, one composition comprising a therapeutically effective amount of pemetrexed and the other comprising a therapeutically effective amount of N-(2-((4-hydroxyphenyl)amino)pyridin-3-yl)-4-methoxybenzenesulfonamide.

Still another embodiment pertains to methods for treating lung cancer with at least additive antitumorigenesis in a mammal, said methods comprising administering thereto therapeutically effective amounts of pemetrexed and N-(2-((4-hydroxyphenyl)amino)pyridin-3-yl)-4-methoxybenzenesulfonamide.

Still another embodiment pertains to a method of treating cancer in a mammal comprising administering therapeutically effective amounts of N-(2-((4-hydroxyphenyl)amino)pyridin-3-yl)-4-methoxybenzenesulfonamide and pemetrexed wherein said N-(2-((4-hydroxyphenyl)amino)pyridin-3-yl)-4-methoxybenzenesulfonamide is dosed orally for least seven continuous days. In another embodiment, said N-(2-((4-hydroxyphenyl)amino)pyridin-3-yl)-4-methoxybenzenesulfonamide is dosed orally for least fourteen continuous days. In another embodiment, said N-(2-((4-hydroxyphenyl)amino)pyridin-3-yl)-4-methoxybenzenesulfonamide is dosed orally for least twenty-one continuous days. In another embodiment, said cancer is selected from lung cancer, breast cancer, prostate cancer, and renal cancer. In another embodiment, said cancer is non-small cell lung cancer. In another embodiment, said cancer is colon cancer. In another embodiment, said mammal is a human, said cancer is non-small cell lung cancer, and said N-(2-((4-hydroxyphenyl)amino)pyridin-3-yl)-4-methoxybenzenesulfonamide is dosed orally for least fourteen continuous days. In another embodiment, said cancer is colon cancer. In another embodiment, said mammal is a human, said cancer is non-small cell lung cancer, and said N-(2-((4-hydroxyphenyl)amino)pyridin-3-yl)-4-methoxybenzenesulfonamide is dosed orally for least twenty-one continuous days. In another embodiment, the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits in combination with pemetrexed.

### DETAILED DESCRIPTION OF THE INVENTION

N-(2-((4-Hydroxyphenyl)amino)pyridin-3-yl)-4-methoxybenzenesulfonamide is also referred to herein as HPM, ABT-751 or 751.

The term “additive antitumorigenesis,” as used herein means greater antitumorigenesis than obtained from use of either N-(2-((4-hydroxyphenyl)amino)pyridin-3-yl)-4-methoxybenzenesulfonamide or a co-therapeutic agent.

The term “Antimetabolites” include ALIMTA® (premetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR, enocitabine, ethnylcytidine, fludarabine, hydroxyurea, 5-fluorouracil (5-FU) alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosate, pelitrexol, pentostatin, raltitrexed, Ribavirin, triapine, trimetrexate, S-1, tiazofurin, tegafur, TS-1, vidarabine, UFT and the like.

The term “antitumorigenesis,” as used herein, means inhibition or reduction of tumor growth.

The term “cancer,” as used herein, means bone marrow dyscrasias, breast (ductal and lobular) cancer, cervical cancer, colon cancer, leukemia, lung (small cell and non-small cell) cancer, lymphoma, melonoma, mouth and tongue cancer, pancreatic cancer, prostate cancer, rectal cancer, renal cancer, sarcoma, stomach cancer, uterine cancer, and cancers resulting from the metastasis of disease from these areas.

The term “continuous,” as used herein, means at least once per day without missing a day.

The term “essentially reduced,” as used herein in reference to severity of an adverse side effect means at least about 50% of the patient population tested did not experience that side effect at the Grade III or IV level, preferably about 75% of the patient population tested did not experience that side effect at the Grade III or IV level, more preferably about 85% of the patient population tested did not experience that side effect at the Grade III or IV level, even more preferably, about 95% of the patient population tested did not experience that side effect at the Grade III or IV level, and most preferably, 100% of the patient population tested did not experience that side effect at the Grade III or IV level.

The term “treating,” as used herein, means at least sustaining and preferably reversing the course of a disease or adverse physiological event.

The term “tumorigenesis,” as used herein, means tumor growth.

The term “drugs of this invention,” as used herein, means antimetabolites and N-(2-((4-hydroxyphenyl)amino)pyridin-3-yl)-4-methoxybenzenesulfonamide.

Drugs of this invention may be administered, for example, orally, parenterally (intramuscularly, intraperintoneally (i.p), intrasternally, intravenously subcutaneously) or transdermally.

Therapeutically effective amounts of drugs of this invention depend on the recipient of treatment, the cancer being treated and severity thereof, compositions containing them, time of administration, route of administration, duration of treatment, their potency, their rate of clearance and whether or not other drugs are co-administered. The amount of a compound of a drug of this invention used to make a composition to be administered daily to a patient in a single dose or in divided doses is from about 0.05 to about 300 mg/kg (mpk) body weight. In another embodiment, the dose of N-(2-((4-Hydroxyphenyl)amino)pyridin-3-yl)-4-methoxybenzenesulfonamide is about 200 mg. Single dose compositions contain these amounts or a combination of submultiples thereof.

Drugs of this invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising drugs of this invention to be administered parenterally or transdermally include, for example, 1,3-butanediol, castor oil, corn oil, cottonseed oil, dextrose, 5% glucose in water (D5W), germ oil, groundnut oil, isotonic sodium chloride solution (0.9% sodium chloride in water), liposomes, oleic acid, olive oil, peanut oil, Ringer's solution, safflower oil, sesame oil, soybean oil, U.S.P. or, water, mixtures thereof and the like.

The term “p.o.” means orally.

The term “q.d.” means once per day.

The term “mpk,” as used herein, means milligrams drug per kilogram mammal.

The term “SEM,” as used herein, means standard error of the mean.

The term “T/C,” as used herein, means size of tumor (treated/control).

The term “s.c.,” as used herein, means subcutaneously.

The term “p-value,” as used herein, means confidence level of comparison to control. For example, a p-value less than 0.5 means having greater than 95% confidence that the result did not occur randomly.

The following examples are presented to provide what is believed to be the most useful and readily understood description of procedures and conceptual aspects of this invention.

### EXAMPLE 1

### Preclinical Non-Small Cell Lung Cancer Study

### HPM and Pemetrexed

Female nu/nu mice (Charles River Labs.) were injected subcutaneously with 0.25 mL of 5×10⁶ Calu-6 cells (1:1 with matrigel) on study day 0. The human Calu-6 cells were obtained from a tumor homogenate from previously inoculated donor mice. The cellular implantation site was the right thigh, and all mice were ear tagged. On day 10, tumors were size matched to approximately 170 mm³ and were placed into the therapy groups outlined in the study design below. The tumors were measured with calipers 2-3 times per week after tumors were palpable. Tumor volume were calculated according to the formula V=L×W²/2 (V: volume, mm³; L: length, mm. W: width, mm). The mice were humanely euthanized when the tumor volumes reached a predetermined size. The study design was as follows:

TABLE 1
Calu-6 Study Design

| Treatment | | | | | |
|---|---|---|---|---|---|
| Groups | N | Agent | mg/kg | Route | Schedule |
| 1 | 10 | ABT-751 | 100 | p.o | Days 10-14 and 20-24; |
| | | | | | 5 days on 5 days off × 2 |
| | | pemetrexed | 0 | i.p | Days 10-14 and Days 20-24 |
| | | Vehicle | | | |
| 2 | 10 | pemetrexed | 150 | i.p | Days 10-14 and 20-24; |
| | | | | | 5 days on 5 days off × 2 |
| | | ABT-751 Vehicle | 0 | i.p | Days 10-14 and Days 20-24 |
| 3 | 10 | ABT-751 | 100 | p.o | Days 10-14 and Days 20-24 |
| | | pemetrexed | 150 | i.p | Days 10-14 and Days 20-24 |
| 4 | 10 | pemetrexed | 150 | i.p | Days 15-19 and Days 25-29; |
| | | | | | 5 days on 5 days off × 2 |
| | | ABT-751 Vehicle | 0 | i.p | Days 10-14 and Days 20-24 |
| 5 | 10 | ABT-751 | 100 | p.o | Days 10-14 and Days 20-24 |
| | | pemetrexed | 150 | i.p | Days 10-14 and 20-24 |
| 6 | 10 | ABT-751 | 100 | p.o | Days 15-19 and Days 25-29 |
| | | pemetrexed | 0 | i.p | Days 10-14 and Days 20-24 |
| | | Vehicle | | | |
| 7 | 10 | ABT-751 | 100 | p.o | Days 15-19 and Days 25-29 |
| | | pemetrexed | 150 | i.p | Days 10-14 and Days 20-24 |
| 8 | 10 | Vehicle | 0 | p.o | Concurrent schedule of |
| | | | | | Group 3 |

ABT-751 was formulated in 1% HCl, 4% ethanol, and 95% D5W while pemetrexed was formulated in normal saline.

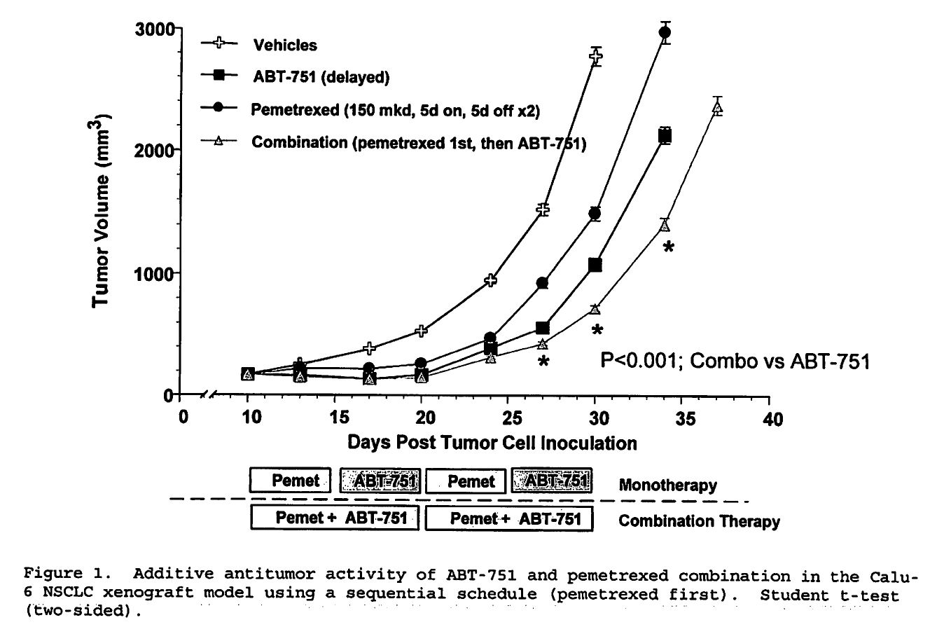

In combination with pemetrexed, ABT-751 demonstrated additive response using a sequential schedule of pemetrexed followed by ABT-751 (Treatment groups #2, 6, 7 and 8). The results are shown in TABLE 2 and FIG. 1.

TABLE 2 shows the In vivo efficacy of ABT-751 with pemetrexed in the Calu-6 flank xenograft model. Study group treatments are defined in Table 1.

TABLE 2

| Treatment | |
|---|---|
| Group | % TGD^{a} |
| 1 | 18.0***^{b} |
| 2 | 11.1** |
| 3 | 18.0*** |
| 4 | 5.6* |
| 5 | 18.0*** |
| 6 | 18.0*** |
| 7 | 26.4*** |
| 8 | 0.0 |

^{a}Mean % TGD (tumor growth delay) increase compared to vehicle in time to 1.5 cc tumor
^{b}p values vs. vehicle,
*<0.05,
**<0.01,
***<0.001

### EXAMPLE 2

### Preclinical Colon Cancer Study

### HPM and Pemetrexed

nu/nu female mice (Charles Rivers Labs) were injected subcutaneously with 0.2 mL of 5×10⁶ GC3TK-100c3 cells (1:1 matrigel) on study day 0. This GC3 human colon carcinoma is thymidine kinase-deficient and pemetrexed is quite sensitive in this model (Schultz, 1999). The cellular implantation site was the right flank, and all mice were ear tagged. Tumors were size matched to 205 mm³, and animals were placed into the therapy groups shown in the study design below (Table 3). The tumors were measured with calipers 2 times per week after tumors were palpable. The mice were humanely euthanized when the tumor volumes reached a predetermined size. The study design was as follows:

TABLE 3
GC3 TK Study Design

| Treatment | | | | | |
|---|---|---|---|---|---|
| Groups | N | Agent | mg/kg | Route | Schedule |
| 1 | 10 | ABT-751 | 100 | p.o | Days 38-42 and 48-52; |
| | | | | | 5 days on 5 days off × 2 |
| | | pemetrexed | 0 | i.p | Days 38-42; q.d. × 5 |
| | | Vehicle | | | |
| 2 | 10 | pemetrexed | 150 | i.p | Days 38-42 |
| | | ABT-751 Vehicle | 0 | i.p | Days 38-42 and 48-52 |
| 3 | 10 | pemetrexed | 150 | i.p | Days 43-47 |
| | | ABT-751 Vehicle | 0 | p.o | Days 38-42 and 48-52 |
| 4 | 10 | ABT-751 | 100 | i.p | Days 38-42 and 48-52 |
| | | pemetrexed | 150 | i.p | Days 38-42 |
| 5 | 10 | ABT-751 | 100 | p.o | Days 38-42 and 48-52 |
| | | pemetrexed | 150 | i.p | Days 43-47 |
| 6 | 10 | Vehicles | — | p.o | Concurrent schedule of |
| | | | | | Group 4 |

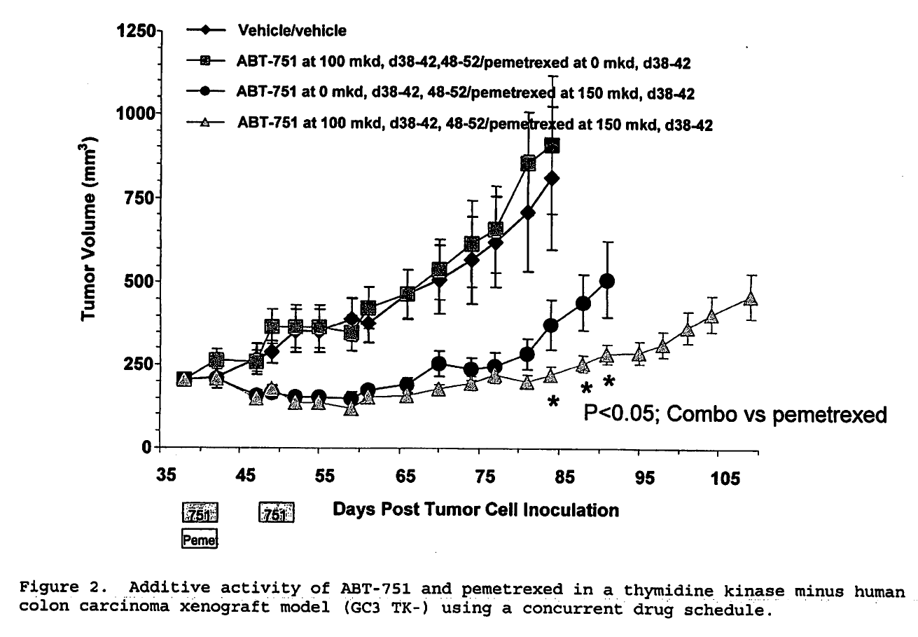

In combination with pemetrexed, ABT-751 demonstrated additive response using a concurrent schedule of pemetrexed and ABT-751 (Treatment groups #1, 2, 4 and 6). The results are shown in TABLE 4 and FIG. 2.

TABLE 4
In vivo efficacy of ABT-751 with pemetrexed in the GC3 TK flank
xenograft model. Study group treatments are defined in Table 3.

| Treatment | |
|---|---|
| Group | % TGD^{a} |
| 1 | 0.0 |
| 2 | 14.8 |
| 3 | 30.7*^{b} |
| 4 | 30.7** |
| 5 | 30.7** |
| 6 | 0.0 |

^{a}Median % TGD (tumor growth delay) increase compared to vehicle in time to 0.75 cc tumor
^{b}p values vs. vehicle,
*<0.05,
**<0.01

### EXAMPLE 3

### Clinical Non-Small Cell Lung Cancer Study

### ABT-751 and Pemetrexed

This study assesses the safety of combining pemetrexed and ABT-751 in subjects with advanced or metastatic NSCLC. The study identified the maximum tolerated dose (MTD) of ABT-751 when administered in combination with pemetrexed at its labeled dose and schedule. Following the determination of the MTD, the study determined if the combination of ABT-751 and pemetrexed improved the progression free survival (PFS) of subjects with NSCLC.

The primary objective of the Phase I portion of this study (conducted in the U.S. only) was to determine the MTD of ABT-751 when administered for 14 consecutive days in a 21-day cycle in combination with standard pemetrexed (500 mg/m²) in subjects with advanced or metastatic NSCLC. The secondary objectives of the Phase I portion of this study was to characterize the pharmacokinetics of ABT-751 and pemetrexed and to assess the safety profile of the ABT-751/pemetrexed combination.

The primary objective of the Phase 2 portion of the study was to assess if the addition of ABT-751 to standard pemetrexed can prolong PFS compared to pemetrexed alone in subjects with advanced or metastatic NSCLC. The secondary objectives of the Phase 2 portion of the study was to determine overall survival, 12-month overall survival, time to disease progression (TTP), disease control rate, response rate, duration of response, quality of life, and characterization of the safety profile of ABT-751 when administered in combination with pemetrexed.

The Phase 1 portion of the study (conducted in the U.S. only) was an open-label dose-escalation to determine the MTD of ABT-751 when administered with pemetrexed in subjects with locally advanced or metastatic refractory NSCLC. A maximum of 20 evaluable subjects were enrolled at up to 6 sites. Once the MTD was reached, the sponsor and investigators were permitted decide to enroll up to twelve additional subjects at the defined MTD of pemetrexed+ABT-751 if additional safety experience was deemed appropriate prior to initiation of the randomized Phase 2 portion of the study.

The Phase 2 portion of the study was double-blind and active-controlled, randomizing subjects in a 1:1 ratio to one of two arms (pemetrexed+oral ABT-751 versus pemetrexed+oral placebo). Approximately 160 subjects were enrolled at up to 50 sites (including the 6 sites from the Phase 1 portion). Overall, the study enrolled approximately 180 subjects and was designed to evaluate the effectiveness of the combination of ABT-751 and pemetrexed on PFS in subjects with advanced or metastatic NSCLC.

All subjects received standard pemetrexed (500 mg/m) on Day 1 of each 21-day cycle, via intravenous infusion over 10 minutes. Oral study drug (ABT-751 or placebo [in the Phase 2 portion only]) was administered orally QD for 14 consecutive days followed by 7 days off drug. Dosing of ABT-751/placebo will occurred with the start of the pemetrexed infusion on Day 1 of each cycle.

Folic acid supplementation began at least 5 days prior to pemetrexed administration. The Screening Visit occurred at least 5 days and not more than 14 days prior to Study Day 1. If the Screening Visit was 8 days or more prior to Study Day 1, the following Screening procedures were repeated on Study Day 1: physical exam, vital signs, laboratory assessments, performance status assessment, and a pregnancy test (for female subjects of child-bearing age). However, if the Screening Visit was 5 to 7 days prior to Study Day 1, only vital signs and a pregnancy test were repeated on Study Day 1.

Baseline radiographic tumor assessments were conducted within 21 days prior to the first dose of ABT-751.

Study visits (including hematology and chemistry laboratory tests) were conducted weekly through Cycles 1 and 2, and then prior to the first dose for all additional cycles administered. Urinalysis tests were performed prior to the first dose of each cycle.

A subject demonstrating a partial response (PR), complete response (CR), or stable disease (SD) continued to receive pemetrexed and ABT-751 or placebo for as long as the subject was deemed to be clinically benefiting from treatment and any side effects manageable. Oral study drug was continued as a single agent in these subjects following the completion of pemetrexed therapy (as determined by the investigator) until disease progression or toxicities prohibit further continuation. In addition, subjects who completed pemetrexed therapy but choose not to continue oral study drug due to intolerability or subjects who discontinued oral study drug due to toxicity remained on study for scheduled tumor assessments until progressive disease was determined or another antitumor therapy initiated.

All subjects had one Follow-up Visit approximately 30 days after the last dose of study drug. If the subject discontinued the study due to toxicities attributable to study drug, additional Follow-up visits were conducted at least every 30 days until the toxicity diminished to an acceptable level or until toxicity was felt to be stable or irreversible.

Radiographic tumor assessments were conducted after every 2 cycles of study drug and/or pemetrexed administration. Response criteria were assessed using the Response Evaluation Criteria in Solid Tumors (RECIST)⁶ to determine response rate, disease control rate, time to progression and PFS. In addition, the investigator evaluated the subject for evidence of disease progression at each visit.

Toxicities were graded at each study visit according to the National Cancer Institute (NCI) Common Terminology Criteria for Adverse Events (CTCAE) Version 3.0.

Subjects had survival assessments every 2 months following study discontinuation of study for a period of up to two years.

A maximum of 20 evaluable subjects were enrolled in the Phase I dose-escalation portion of the study, which was conducted in the U.S. only. The initial dose of ABT-751 was 200 mg QD. Doses of ABT-751 escalated by 50 mg increments and dose reduction occurred in 25 mg decrements.

All subjects were included for analysis of safety data. Subjects in the Phase I portion of the study who were not evaluable were replaced. Evaluable subjects were defined as those subjects who:
Experience a DLT, or
Complete at least one cycle (21 days) of dosing with >80% compliance on the 14-day dosing schedule.

Blood sampling for pharmacokinetic analysis was conducted on Cycle 1, Day 1, at O-hr (pre-dose), immediately after the end of the 10-minute pemetrexed infusion, and following ABT-751 administration at 0.5, 1, 2, 4, 6, 8, and 24 hours (prior to ABT-751 dosing on Study Day 2).

After the MTD was determined, the Phase 2 portion of the study randomized 160 subjects from approximately 50 sites in a 1:1 ratio to either pemetrexed+ABT-751 (80 subjects) or pemetrexed+placebo (80 subjects). All participating sites were informed by Abbott of the MTD established in the Phase I portion of the study prior to enrollment of subjects in the Phase 2 portion of the study. Subjects received either oral ABT-751 or oral placebo on Days 1-14 of each 21-day cycle. All subjects received pemetrexed on Day I of each cycle.

Subjects completed a quality of life questionnaire (FACT-L; Appendix D) at Screening, on Day 1 of each cycle, at the Final Visit and approximately 30 days following completion of therapy.

For those subjects in the U.S. who consented, pharmacodynamic (PD) samples for analysis of circulating tumor cells (CTCs) were collected at Screening, after Cycle 1, and at the final visit. Pharmacodynamic samples for proteomic analysis were collected for all consenting subjects at Screening, after Cycles 1 and 2, and at the Final Visit.

The date when the 110th confirmed event of progression occurred or 3 months after last patient enrolled, whichever came later, was defined as the primary data “cutoff” date for the efficacy and safety analyses, and safety and efficacy data up to and including this date will be collected prior to breaking the blind. During this data collection period, active subjects continued to receive blinded study drug and pemetrexed, if applicable.

When the data collection period was completed the study blind will be broken. Active subjects had a Final Visit and discontinued the study, at which point the study (M05-780) was completed and all remaining data, with the exception of overall survival, was collected and entered into the clinical database.

Overall survival was collected on all subjects for up to 2 years after they discontinued the study.
Controls in the above examples comprised vehicle only.

The term “p.o.” means orally.

The term “q.d.” means once per day.

The term “mpk,” as used herein, means milligrams drug per kilogram mammal.

The term “SEM,” as used herein, means standard error of the mean.

The term “T/C,” as used herein, means size of tumor (treated/control).

The term “s.c.,” as used herein, means subcutaneously.

The term “p-value,” as used herein, means confidence level of comparison to control. For example, a p-value less than 0.5 means having greater than 95% confidence that the result did not occur randomly.

The foregoing is meant to illustrate the invention and not limit it to the disclosed embodiments. Variations and changes obvious to one skilled in the art are intended to be within the scope and nature of the invention as defined in the claims.

We claim:

1. A composition for treating cancer in a mammal comprising therapeutically effective amounts of an antimetabolite and N-(2-((4-hydroxyphenyl)amino)pyridin-3-yl)-4-methoxybenzenesulfonamide.

2. A composition for treating cancer in a mammal comprising therapeutically effective amounts of pemetrexed and N-(2-((4-hydroxyphenyl)amino)pyridin-3-yl)-4-methoxybenzenesulfonamide.

3. A method of treating cancer in a human comprising administering therapeutically effective amounts of pemetrexed and N-(2-((4-hydroxyphenyl)amino)pyridin-3-yl)-4-methoxybenzenesulfonamide.

4. A method of treating cancer in a mammal comprising administering therapeutically effective amounts of N-(2-((4-hydroxyphenyl)amino)pyridin-3-yl)-4-methoxybenzenesulfonamide and pemetrexed wherein the N-(2-((4-hydroxyphenyl)amino)pyridin-3-yl)-4-methoxybenzenesulfonamide is dosed orally for least seven continuous days.

5. The method of claim 4 wherein the mammal is a human.

6. The method of claim 5 wherein the cancer is selected from lung cancer, breast cancer, prostate cancer, and renal cancer.

7. The method of claim 6 wherein the N-(2-((4-hydroxyphenyl)amino)pyridin-3-yl)-4-methoxybenzenesulfonamide is dosed orally for least fourteen continuous days.
